(19) United States
(12) Patent Application Publication
(10) Pub. No.: US 2012/0129706 A1
(43) Pub. Date: May 24, 2012

(54) Method of Assessing Soil Quality and Health

(76) Inventors: Ashvini Chauhan, Tallahassee, FL (US); Dawn Erica Lewis, Tallahassee, FL (US)

(21) Appl. No.: 13/299,502

(22) Filed: Nov. 18, 2011

Related U.S. Application Data
(60) Provisional application No. 61/415,905, filed on Nov. 22, 2010.

(51) Int. Cl. C40B 30/00; C12Q 1/68

(52) U.S. Cl. 506/7; 435/6.12

(57) ABSTRACT

The present invention relates to a method of assessing the soil quality and health of mined and un-mined lands. The present invention further relates to a method of assessing ecosystem health and soil restoration efforts. The method employs a multi-prong approach combining DNA fingerprinting and in-depth pyrosequencing of soil microorganisms that are responsible for maintaining soil fertility and productivity. The method incorporates assessing the indigenous bacterial community structure data derived from PCR-DGGE analysis and 165 rRNA gene clone libraries, and then further identification of those microorganisms that are known to promote soil health by soil metagenome analysis. Disparity between un-mined and post-mined soils are comprehensively studied via multivariate statistical analyses on the polyphasic dataset.

### CROSS-REFERENCE TO RELATED APPLICATIONS

This Application claims the benefit of Provisional application No. 61/415,905 filed on Nov. 22, 2010.

### FEDERALLY SPONSORED RESEARCH

Not applicable

### SEQUENCE LISTING OR PROGRAM

Not applicable

### FIELD OF INVENTION

The present invention relates to a method of assessing the soil quality and health of mined and un-mined lands. The present invention further relates to a method of assessing ecosystem health and soil restoration efforts. The method employs a multi-prong approach combining DNA fingerprinting and in-depth pyrosequencing of soil microorganisms that are responsible for maintaining soil fertility and productivity. The method incorporates assessing the indigenous bacterial community structure data derived from PCR-DGGE analysis and 165 rRNA gene clone libraries, and then further identification of those microorganisms that are known to promote soil health by soil metagenome analysis. Disparity between un-mined and post-mined soils are comprehensively studied via multivariate statistical analyses on the polyphasic dataset.

### BACKGROUND OF INVENTION

It is generally well-known that the stability and function of a soil ecosystem depends on the cycling of nutrients by soil microorganisms. Microbial communities are known to play a key role in the maintenance of soil health and quality, and rapidly adapt to environmental change. Miniscule shifts in microbial populations and activities can serve as a sensitive bio-indicator of ecosystem health. Hence, microbial indicators can provide precise and quantifiable assessment of ecological change and can be used with complementing physical and chemical properties as suitable indicators of soil health and quality.

Soil health refers to the biological, chemical, and physical features of soil that are essential to long-term, sustainable agricultural productivity with minimal environmental impact. Soil health provides an overall picture of soil functionality. Although it cannot be measured directly, soil health can be inferred by measuring specific soil properties (e.g. organic matter content) and by observing soil status (e.g. fertility).

One of the key objectives in determining soil health is to acquire indicators that can be used to evaluate the soil's current status and thereafter be used to develop sustainable agricultural systems. In this regard, significant progress has been made over the last few years in the development of specific bio-markers and macromolecular probes, enabling rapid and reliable measurements of soil microbial communities. In addition, modern molecular biological techniques, such as fluorescence in situ hybridization (FISH), reverse transcriptase polymerase chain reaction (RT-PCR), denaturing gradient gel electrophoresis (DGGE), and terminal restriction fragment length polymorphism (T-RFLP) have facilitated the analysis of microbial biodiversity and activity, whereas the application of modern analytical techniques, such as nuclear magnetic resonance (NMR) and pyrolysis-gas chromatography-mass spectrometry (Py-GC-MS), have provided data on soil chemistry.

Perhaps there is no other area where such efforts of determining soil health status are more important, than in assessing the restoration efforts of mined lands. Changes in soil microbial community structure can provide early signs of environmental degradation and potentially predict the course of restoration. The present invention concentrates and relates to restoration of stripped mined lands, such as in the case of bauxite mining.

Mining of bauxite rich soils involves striping the vegetation and stock-piling of the top 0-30 cm soils alongside the mined area. Once the soils are depleted in bauxite ore, rehabilitation is performed by reshaping the area using the stock-piled soil, followed by planting of grass and other crops. The general shortage of agriculturally viable land, as in Jamaica, has spiked an immense interest to rehabilitate and use post-mined lands for agricultural purposes. However, mining regulations often require bauxite industries to restore mined lands such that their productivity mirrors those found in un-mined lands. Nevertheless, because mining is a temporary change in land use, criteria of successful restoration prior to public usage remains obscure. Regardless of this fact, this temporary land usage has been shown to cause long lasting impacts to soil geochemical functioning. Large-scale failure of crops is largely attributed to acute nutrient stress and increased susceptibility to soil-borne pathogens, leading to reduction in exports and contributing further to socio-economic tragedies.

One of the perceived challenges of the reclamation of stripped mined lands is the development of effective land and soil restoration practices so that mined land can be returned to a state where agriculture can be produced. Prior art teaches that there are distinct differences in the physico-chemical and biological properties of native soil and post-mined reclaimed soils. However, because the structure and associated loss in the functions of biota in bauxite mined soils remain poorly understood, establishing a restoration trajectory for such anthropogenically altered soils is complex. Moreover, previous studies have focused on changes in animal or plant communities as indicators of ecosystem level changes following an anthropogenic disturbance. Nevertheless, there is a perceived difficulty in understanding the variations in soils in the absence of knowledge derived from both chemical and biological approaches due to the fact that microorganisms affect the environment and vice versa.

The present invention departs from the prior art by focusing on the changes in microbial activities, structure, and functions which can precede detectable changes in soil physio-chemical or ecological integrity. The present invention assesses microbial community shifts resulting from immigration and emigration processes during stripping and replanting activities that are typically involved during strip mining operations, such as bauxite mining; The present invention further departs from the prior art by presenting a multi pronged approach for the assessment of soil quality by assessing the indigenous bacterial community structure data derived from PCR-DGGE analysis and 165 rRNA gene clone libraries, and then further identification of those microorganisms that are known to promote soil health by soil metagenome analysis. Disparity between un-mined and mined soils are then comprehensively studied via multivariate statistical analyses on the polyphasic dataset.

### BRIEF SUMMARY OF THE INVENTION

The present invention relates to a method of assessing soil quality and health of mined and un-mined lands. The present invention further relates to a method of assessing ecosystem health and soil restoration efforts of stripped mined lands. The method employs a multi-prong approach combining DNA fingerprinting techniques with more advanced metagenome analysis.

In one aspect of the invention, soil samples are collected from at least one un-mined site and at least one mined site. All samples are analyzed to assess the disparity between un-mined and mined lands as it relates to those microorganisms that are affected by mining activities. Samples are analyzed via DNA fingerprinting and advanced molecular biology techniques to obtain microbial data. Geochemical data is also obtained from the samples. The resulting geochemical data and microbial data are then analyzed via a plurality of multivariate statistical analyses including ANOVA, Tukey test, principal component analysis (PCA), Cluster analysis, Shannon-Weaver index of diversity, Equitability index, P-test, UniFrac metric test, lineage-specific analysis, G-test, and normalized ratios. The resulting data is then compared to assess any perceived differences between and among the microbial communities of un-mined and mined lands to obtain an indicator of soil quality and health.

In another aspect of the invention, samples are collected from a plurality of chronosequence mined sites that have been undergoing a rehabilitation process. Samples may also be pooled in order to streamline the collection of geochemical data and microbial data.

### BRIEF DESCRIPTION OF DRAWINGS

Not applicable

### DETAILED DESCRIPTION OF THE INVENTION

The present invention is directed towards a method of determining and assessing the mining impacts and rehabilitation success of stripped mined lands, as in bauxite mining, by analyzing bacterial community structure within un-mined and mined lands.

In the preferred embodiment, a plurality of soil samples are collected from at least one un-mined site, and a plurality of soil samples are collected from a plurality of mined sites. All samples are then analyzed to obtain geochemical data by the normal and customary means. As claimed herein, such geochemical data may comprise said samples pH levels and moisture content. Molecular fingerprinting is also performed on all samples to obtain microbial data. Molecular fingerprinting may be performed by the normal and customary means, comprising, as claimed herein, extracting the genomic DNA from each of the samples and then amplifying the bacterial 16S rDNA via polymerase chain reaction (PCR) and thereafter performing denaturing gradient gel electrophoresis (DGGE) to produce banding patterns or fingerprints of the soil microbia. Molecular fingerprinting further comprises quantitative PCR analysis on the 16S ribosomal genes and advanced pyrosequencing, and the construction of 16S rDNA clone libraries.

The resulting geochemical data and microbial data are then analyzed via a plurality of multivariate statistical analyses to produce statistical data results, i.e. polyphasic dataset. The results from the multivariate statistical analyses on the polyphasic dataset are then compared to assess any perceived differences between and among the microbial communities of un-mined and mined lands to obtain an indicator of soil quality and health.

In further describing the claimed invention, as claimed herein, the geochemical data is analyzed via the analysis of variance (ANOVA), the Tukey test, and principal component analysis (PCA). Cluster analysis is also performed via the unweighted pair group method with arithmetic mean (UPGMA) to produce linkage dendrograms.

As claimed herein, the microbial data is analyzed via the Shannon-Weaver index of diversity, equitability index, one-way ANOVA, and UPGMA to generate a dendrogram. The microbial data is further analyzed via the P-test, UniFrac metric test, principal component analysis (PCA), lineage-specific analysis, G-test, and normalized ratios.

In an alternative embodiment, the plurality of mined site samples further comprise a chronosequence of mined sites adjacent to the un-mined site. A chronosequence represents variable time series of soils undergoing ecological succession and facilitates assessment of soil degradation or improvement by comparing the physico-chemical and biological properties at different rehabilitation ages. In the alternative embodiment, the method further comprises pooling more than one of the plurality of samples taken from the un-mined site, and pooling more than one of the plurality of samples taken from the plurality of mined sites to form at least one pooled un-mined sample and at least one pooled mined sample, respectively. The pooled un-mined sample and pooled mined sample are further subjected to molecular fingerprinting to obtain microbial data. As described herein, molecular fingerprinting may be performed by the normal and customary means, including as claimed herein extracting DNA from the pooled un-mined sample and the pooled mined sample. The resulting 16Sr DNA genes are then amplified, cloned, and then sequenced. Quantitative PCR analysis on the 16S ribosomal genes and advanced pyrosequencing are also performed.

With respect to the employment of the claimed invention, analyses, as described herein, coupled with rRNA gene sequencing, pyrosequencing, and quantitative PCR analysis demonstrated that bauxite mining activities, even after two decades of cessation, negatively impacted soil microbial properties, including a significant decrease in bacterial diversity, and a 3-log decrease in rRNA gene abundance. Specifically, proteobacterial and acidobacterial rRNA abundances decreased in all post-mined soils. Conversely, the relative abundance of Firmicutes were higher in post-mined soils compared with un-mined soils. Resulting data indicated that mining activities cause long-lasting perturbances to the soil microbiota and potentially their associated geochemical functions.

We claim:

1. A method of assessing soil quality and health comprising:
a. Obtaining at least one sample from at least one mined site;
b. Obtaining at least one sample from at least one un-mined site;
c. Analyzing all samples to produce geochemical data;
d. Employing a means of molecular fingerprinting all samples to produce microbial data;
e. Analyzing said geochemical data and microbial data via a plurality of multivariate statistical analyses to produce statistical data results; and
f. Analyzing said statistical data results to assess any perceived differences between and among the microbial communities of un-mined and mined lands to obtain an indicator of soil quality and health.

2. The method of claim 1, wherein said means of molecular fingerprinting further comprises:
a. Extracting genomic DNA from all samples to produce bacterial 16S rDNA;
b. Amplifying said bacterial 16S rDNA via polymerase chain reaction to produce copies of bacterial 16S ribosomal genes; and
c. Performing denaturing gradient gel electrophoresis on said copies of the bacterial 16S ribosomal genes to produce banding patterns.

3. The method of claim 2, wherein said geochemical data is analyzed via the analysis of variance test, the Tukey test, principal component analysis, and cluster analysis.

4. The method of claim 3, wherein said microbial data is analyzed via that Shannon-Weaver index of diversity, the equitability index, one-way analysis of variance, and the unweighted pair group method with arithmetic mean test.

5. The method of claim 4, wherein said geochemical data includes pH levels and moisture content.

6. The method of claim 5, wherein said mined site and said un-mined site are adjacent to each other.

7. The method of claim 6, wherein said mined site is a bauxite mined site.

8. A method of assessing soil quality and health comprising:
a. Obtaining more than one sample from a plurality of mined sites;
b. Obtaining more than one sample from at least one un-mined site;
c. Analyzing all samples to obtain geochemical data;
d. Employing a means of molecular fingerprinting all samples to produce microbial data;
e. Analyzing said geochemical data via a plurality of multivariate statistical analyses;
f. Analyzing said microbial data via a plurality of multivariate statistical analyses; and
g. Analyzing the results from said multivariate statistical analyses to any perceived differences between and among the microbial communities of un-mined and mined lands to obtain an indicator of soil quality and health.

9. The method of claim 8, wherein said method further comprises:
a. Pooling the samples from said plurality of mined sites to form at least one pooled mined sample; and
b. Pooling the samples from said un-mined site to form at least one pooled un-mined sample.

10. The method of claim 9, wherein said means of molecular fingerprinting further comprises:
a. Extracting genomic DNA from all samples to produce bacterial 16S rDNA;
b. Amplifying said bacterial 16S rDNA via polymerase chain reaction to produce copies of bacterial 16S ribosomal genes;
c. Performing denaturing gradient gel electrophoresis on said copies of the bacterial 16S ribosomal genes to produce banding patterns;
d. Cloning of the 16S rDNA obtain from said pooled un-mined sample and said pooled mined sample;
e. Advanced pyrosequencing of the soil metagenome of all samples; and
f. Performing quantitative PCR analysis on all samples.

11. The method of claim. 10, wherein the step of analyzing said geochemical data via a plurality of multivariate statistical analyses further comprises:
a. Performing the analysis of variance test;
b. Performing the Tukey test;
c. Performing principal component analysis; and
d. Performing cluster analysis.

12. The method of claim 11, wherein the step of analyzing said microbial data via a plurality of multivariate statistical analyses further comprises:
a. Performing the Shannon-Weaver index of diversity;
b. Performing the equitability index;
c. Performing one-way analysis of variance;
d. Performing the unweighted pair group method with arithmetic mean test;
e. Performing the P-test;
f. Performing the UniFrac metric test;
g. Performing principal component analysis;
h. Performing lineage specific analysis;
i. Performing the G-test; and
j. Performing normalized ratios.

13. The method of claim 12, wherein said mined site and said un-mined site are adjacent to each other.

14. The method of claim 13, wherein said mined sites are a chronosequence of mined sites.

15. The method of claim 14, wherein said chronosequence of mined sites are bauxite mined sites.

16. A method of assessing soil quality and health comprising:
a. Obtaining a plurality of samples from a plurality of chronosequence bauxite mined sites;
b. Obtaining a plurality of samples from at least one un-mined site, said un-mined site being adjacent to the said plurality of bauxite mined sites;
c. Pooling the samples from said plurality of chronosequence mined sites to form at least one pooled mined sample;
d. Pooling the samples from said un-mined site to form at least one pooled un-mined sample;
e. Employing a means of analyzing all samples to obtain geochemical data, said geochemical data comprises pH levels and organic matter content;
f. Employing a means of molecular fingerprinting all samples to produce microbial data;
g. Analyzing said geochemical data via a plurality of multivariate statistical analyses comprising the analysis of variance test, the Tukey test, principal component analysis, and cluster analysis;
h. Analyzing said microbial data via a plurality of multivariate statistical analyses comprising the Shannon-Weaver index of diversity test, the Equitability Index test, one-way analysis of variance test, the unweighted pair group method with arithmetic mean test, the P-test, the UniFrac metric test, principal component analysis, lineage specific analysis, the G-test, and normalized ratios; and
i. Analyzing the results from said multivariate statistical analyses to determine any perceived differences between and among the microbial communities of un-mined and mined lands to obtain an indicator of soil quality and health.

17. The method of claim 16, wherein said means of molecular fingerprinting further comprises:
a. Extracting genomic DNA from all samples to produce bacterial 16S rDNA, amplifying said bacterial 16S rDNA via polymerase chain reaction to produce copies of bacterial 16S ribosomal genes;
b. Performing denaturing gradient gel electrophoresis on said copies of the bacterial 16S ribosomal genes to produce banding patterns;
c. Cloning of the 16S rDNA obtain from said pooled un-mined sample and said pooled mined sample;
d. Advanced pyrosequencing of the soil metagenome of all samples; and
e. Performing quantitative PCR analysis on all samples.
